(19) United States
(12) Patent Application Publication
(10) Pub. No.: US 2016/0106514 A1
(43) Pub. Date: Apr. 21, 2016

(54) MEDICAL IMAGING APPARATUS WITH IMAGE PROJECTING UNITS

(71) Applicant: GENERAL ELECTRIC COMPANY, Schenectady, NY (US)

(72) Inventors: Ritesh Saxena, Bangalore (IN); Chinmoy Goswami, Waukesha, WI (US); Jason Lee Philips, Waukesha, WI (US); Yasuyuki Innami, Hino-shi (JP); Anh-Kiet Dam, Waukesha, WI (US)

(21) Appl. No.: 14/893,867

(22) PCT Filed: May 27, 2014
(86) PCT No.: PCT/US2014/039514

§ 371 Date: Nov. 24, 2015

(30) Foreign Application Priority Data
May 29, 2013 (IN) 2340/CHE/2013

(51) Int. Cl. H04N 7/18; G06F 3/048; H04N 5/225; H04N 9/31

(52) U.S. Cl. CPC A61B 19/5225; H04N 5/225; H04N 9/31; G06F 3/048; A61M 2021/005

(57) ABSTRACT

A medical imaging apparatus having multiple image projecting units for projecting images for a subject to view is disclosed. The medical imaging apparatus is present in a scanning room for performing imaging of the subject. The medical imaging apparatus includes a table for holding the subject. The table along with the subject is facilitated by an image capturing subsystem to pass therethrough for capturing medical images of the subject. The multiple image projecting units are configured within the image capturing subsystem for projecting images to one or more of walls of the scanning room and an inner surface of the image capturing subsystem.

### CROSS-REFERENCE TO RELATED APPLICATIONS

This is a national stage application under 35 U.S.C. §371(c) of prior filed, co-pending PCT Patent Application No. PCT/US2014/039514, filed on May 27, 2014, which claims priority to India Patent Application No. 2340/CHE/2013, filed on May 29, 2013. The aforementioned applications are herein incorporated in their entirety by reference.

The subject matter disclosed herein relates to medical imaging. More specifically the subject matter relates to a medical imaging apparatus for projecting images from image projecting units in the medical imaging apparatus.

### BACKGROUND

Medical imaging apparatus are used in different applications to generate images of different regions or areas (e.g. different organs) of patients or other objects. Different types of medical imaging apparatus are available and they include for example, ultrasound imaging system, X-ray system, computed tomography system, single photon emission computed tomography system, magnetic resonance imaging system, or the like. Considering the case of a magnetic resonance imaging system, this system generates more noise that may be distracting to a subject undergoing the imaging procedure. If the subject is a child the noise may cause panic for the child which may be more alarming. The sounds may also create anxiety in the subject resulting in subject uncomfortably moving while performing the imaging process. These movements affect the quality of medical images captured. The movements can also cause injuries to the subject and the imaging process may get prolonged due to the need of performing multiple attempts to capture the correct or better medical images. Conducting the imaging process multiple times also annoys the technician and the subject.

Thus there is a need for a medical imaging apparatus for imaging the subjects and reducing the distractions to the subject during imaging procedure.

### BRIEF DESCRIPTION

The above-mentioned shortcomings, disadvantages and problems are addressed herein which will be understood by reading and understanding the following specification.

As discussed in detail below, embodiments of the invention include a medical imaging apparatus having multiple image projecting units for projecting images for a subject to view is disclosed. The medical imaging apparatus is present in a scanning room for performing imaging of the subject. The medical imaging apparatus includes a table for holding the subject. The table along with the subject is facilitated by an image capturing subsystem to pass therethrough for capturing medical images of the subject. The multiple image projecting units are configured within the image capturing subsystem for projecting images to one or more of walls of the scanning room and an inner surface of the image capturing subsystem.

In another embodiment an image projection system coupled to a medical imaging apparatus is disclosed. The image projection system comprises an image projection module for controlling projection of images for a subject to view. One or more images are projected to one or more of walls of a scanning room and an inner surface of an image capturing subsystem of the medical imaging subsystem. The image capturing subsystem is configured to facilitate the subject to pass therethrough for capturing medical images of the subject. One or more memory(s) may be communicably coupled to the image projection module.

### BRIEF DESCRIPTION OF THE DRAWINGS

FIG. 1 illustrates a medical imaging apparatus for performing an imaging procedure on a subject;

FIG. 2 is a schematic illustration a table capable of holding a subject in accordance with an embodiment;

FIG. 3 is a medical imaging apparatus having multiple image projecting units for projecting images for subject to view in accordance with an embodiment;

FIG. 4 schematically illustrates a technician monitoring a subject present in the medical imaging apparatus and controlling the medical imaging apparatus in accordance with an embodiment;

FIG. 5 illustrates a system for managing the function of the image projecting units present in a medical imaging apparatus in accordance with an embodiment;

FIG. 6 is a schematic illustration of a user interface (UI) presenting images for example movie during a scanning or imaging procedure and when scanning or imaging procedure is not performed in accordance with an exemplary embodiment;

FIG. 7 is a schematic illustration of a UI visually providing instructions to the subject while performing an imaging or scanning procedure in accordance with an exemplary embodiment;

FIG. 8 illustrates a UI presenting a message “wait for a while” along with a movie when the subject enters the scanning room in accordance with an exemplary embodiment;

FIG. 9 illustrates a UI displaying a face of a technician in accordance with an exemplary embodiment; and

FIG. 10 illustrates a block diagram of an image projection system in accordance with an embodiment.

### DETAILED DESCRIPTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

To the extent that the figures illustrate diagrams of the functional blocks of various embodiments, the functional blocks are not necessarily indicative of the division between hardware circuitry. One or more of the functional blocks (e.g., processors or memories) may be implemented in a single piece of hardware (e.g., a general purpose signal processor or random access memory, hard disk, or the like) or multiple pieces of hardware. Similarly, the programs may be standalone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

A medical imaging apparatus having multiple image projecting units for projecting images for a subject to view is disclosed. The medical imaging apparatus is present in a scanning room for performing imaging of the subject. The medical imaging apparatus includes a table for holding the subject. The table along with the subject is facilitated by an image capturing subsystem to pass therethrough for capturing medical images of the subject. The multiple image projecting units are configured within the image capturing subsystem for projecting images to one or more of walls of the scanning room and an inner surface of the image capturing subsystem.

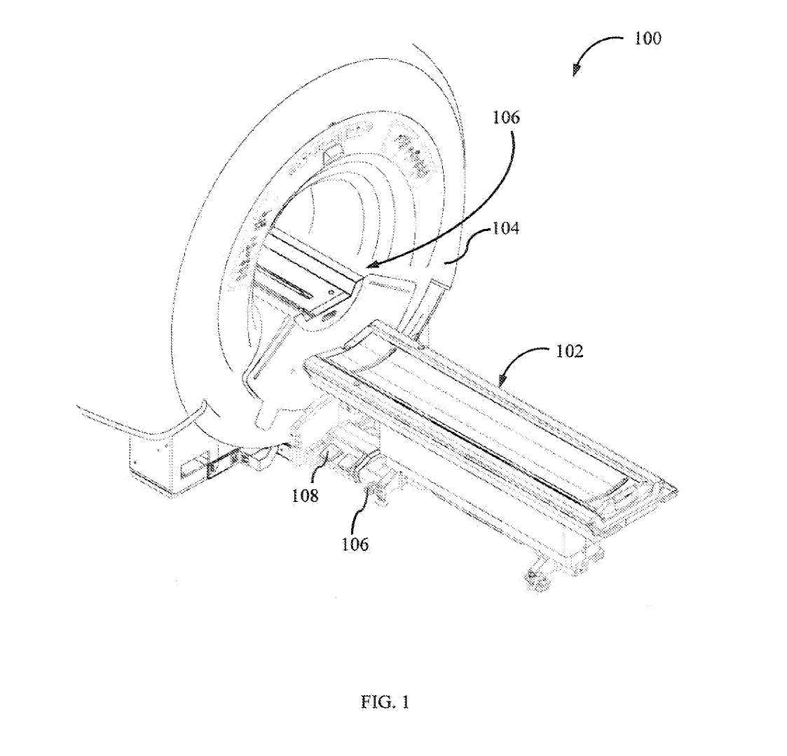

Various embodiments of the invention provide a medical imaging apparatus 100 as shown in FIG. 1. FIG. 1 illustrates the medical imaging apparatus 100 that is used for performing imaging of an object, typically a human body in accordance with an embodiment. The medical imaging apparatus 100 includes for example, a computed tomography imaging system, a single photon emission computed tomography imaging system, a magnetic resonance imaging system, a positron emission tomography system or the like. Multiple images of the body are captured to analyze tissues and structures of the body. The medical imaging apparatus 100 includes a table 102 that can be docked to an image capturing subsystem 104 (also known as a bore or a gantry). A table such as the table 102 includes a cradle, cradle rollers facilitating movement of the cradle, a table top positioned on the cradle, table vertical movement assembly (for example scissor units), handles for moving the table, motors, and other electronic/electrical units for controlling the function of the table. Before docking the cradle, the table top may be in a lowered position. When docked the subject can lie down on the table 102. Thereafter the cradle of the table 102 is lifted in a vertical direction using the table vertical movement assembly to align the cradle with respect to the gantry of the medical imaging apparatus 100. The table vertical movement assembly may be operated using foot switches such as a foot switch 106, and a foot switch 108. The foot switch 106 may be used to vertically move the cradle along with the table top in the upward direction. Whereas the foot switch 108 may be used to vertically move down the cradle along with the table top. The cradle is then moved in a horizontal direction for allowing the subject present on the table top to move into the gantry for performing the imaging of the subject at the image capturing subsystem 104. The table described herein is according to an exemplary embodiment and hence it may be appreciated that other tables can be used in a medical imaging apparatus.

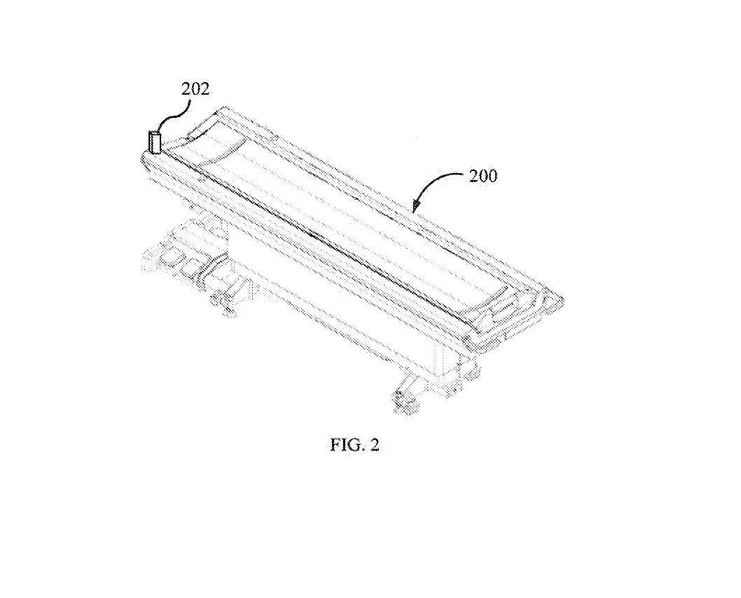

FIG. 2 illustrates a table 200 capable of holding a subject in accordance with an embodiment. The table 200 can be used to position the subject with respect to an image capturing subsystem (such as the image capturing subsystem 104) for capturing medical images associated with the subject. For example, in the case of a magnetic resonance (MR) image capturing subsystem, the table 200 can position the subject so as to capture multiple MR images from the subject. As explained previously the sounds generated from the MR image capturing subsystem may be alarming and disturbing for the subject, hence an image projecting unit 202 is provided on the table 200. The image projecting unit 202 receives images from an image source. The image source may be directly or indirectly connected to the image projecting unit 202. The images may include, but are not limited to, video images, static images, animated images and procedural images. The images may be two dimensional images and three dimensional images. In case of the three dimensional images the subject may use a three dimensional viewing goggles for viewing the images. The images projected may be live images or pre-stored images. The image projecting unit 202 receives the images and projects them for the subject to view. These presented images diverts the attention of the subject from the sounds produced and thereby reduce disturbances caused to the subject. As discussed earlier the table 200 is used to hold and position the subject with respect to the image capturing subsystem, so the image projecting unit 202 may project the images to an inner surface of the image capturing subsystem.

FIG. 3 illustrates a medical imaging apparatus 300 having multiple image projecting units for projecting images for subject to view in accordance with an embodiment. The medical imaging apparatus 300 is placed in a scanning room 302. The scanning room 302 has multiple walls such as a wall 304, a wall 306 and a wall 308. The subject may be instructed to enter the scanning room 302 by a technician for example an MR technician for scanning or imaging. In an embodiment the medical imaging apparatus 300 may include three image projecting units for instance an image projecting unit 310, an image projecting unit 312, an image projecting unit 314 and the image projecting unit 202. The image projecting units may be configured so as to project the images to the walls of the scanning room and the inner surface of an image capturing subsystem 316. Thus the image projecting unit 310, the image projecting unit 312 and the image projecting unit 314 are configured in the image capturing subsystem 316 as shown in FIG. 3. The image projecting unit 310 can project images 318 on to the wall 304 and the image projecting unit 312 can project images 320 on to the wall 308. Further the image projecting unit 314 projects images 322 on to a ceiling 324 of the scanning room 302. When a subject enters the scanning room the images may be presented or projected on to the walls 304 and 308 and the ceiling 324. These images attract the attention of the subject and deviates the subject's focus from the sounds generated from the medical imaging apparatus 300. For instance if the subject is a child then videos interesting to the child such as cartoons, space adventure and forest safari may be displayed. In an embodiment all the image projecting units 310, 312 and 314 may project the images at the same time. In another instance these imaging projecting units may function to project the images at different time. The operation of the image projecting units 310, 312 and 314 may be controlled by an operator or the technician.

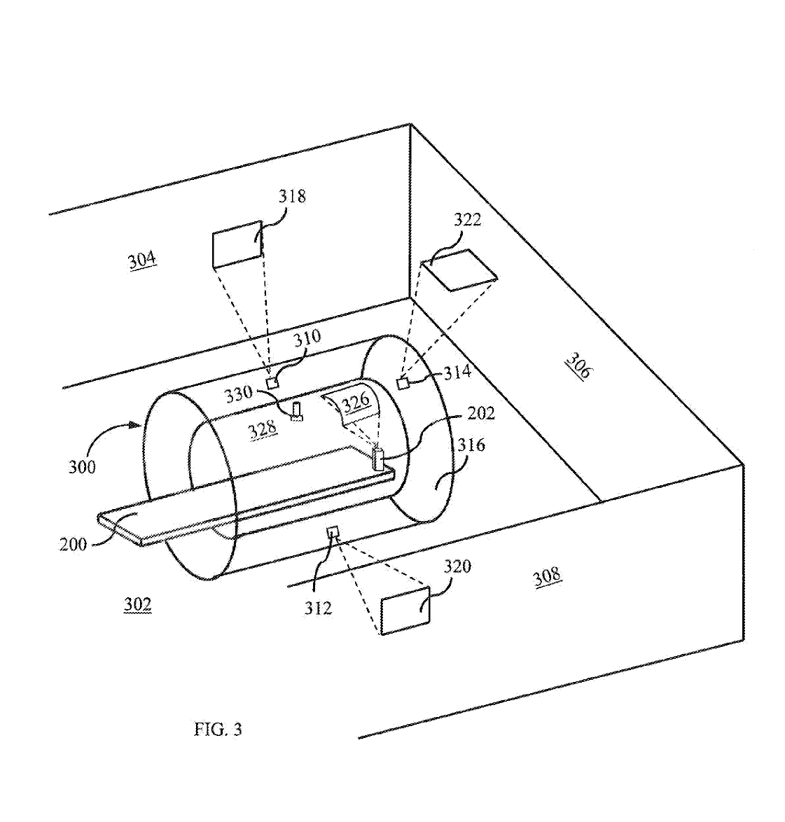

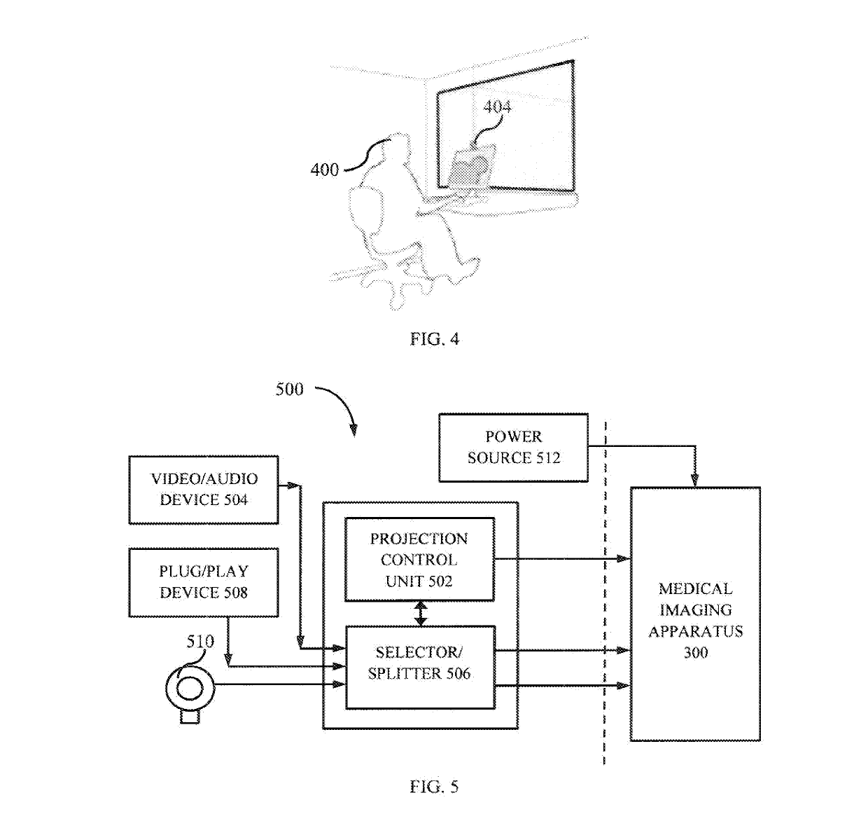

When an imaging procedure such as a MR image needs to be taken of the subject, the subject may lie down on the table 200 and the table 200 is allowed to move into the image capturing subsystem 316. As the subject passes through the image capturing subsystem 316 the images need to be projected in a convenient manner as the images projected on the walls 304 and 308 and the ceiling 324 may not be visible. To this end the image projecting unit 202 present on the table 200 may project images 326 on to an inner surface 328 of the image capturing subsystem 316. FIG. 3 depicts the images 326 are projected on an upper portion of the inner surface 328 however it may be appreciated that the image projecting unit 202 may be configured to project the images 326 at any portion of the inner surface 328. When the subject lies on the table 200 the subject may be conveniently engaged in viewing the images 326. As a result, anxiety of the subject while imaging can be reduced and unwanted movements by the subject can be also avoided. The functioning of the image projecting units 310, 312 and 314 can be controlled by an operator or a technician 400 present in a room located external or internal to the scanning room 302 as shown in FIG. 4. Further it may be noted that the medical imaging apparatus 300 is shown to include three image projecting units as an exemplary embodiment, however the medical imaging apparatus 300 may include more image projecting units for example an image projecting unit may be configured in the image capturing subsystem 316 to project images to the floor of the scanning room 302. As the image projecting units are all configured within or inbuilt in the image capturing subsystem 316, any modifications to the scanning room 302 are avoided.

The subject passing through the image capturing subsystem 316 may be monitored using an imaging device 330 such as a video camera and a still camera. The imaging device 330 captures the subject and present to the technician 400 through a device 402. The imaging device 330 can be used to monitor the movements of the subject and assists the technician 400 to give instructions to the subject. The instructions may be associated with performing imaging process and also notify the subject of any unwanted movements that affects the imaging process. These movements of the subject can affect the quality of images captured. As shown in FIG. 4, an imaging device 404 may capture the technician 400 (for instance a face of the technician 400) and present to the subject through the imaging projecting units. This is explained further in conjunction with FIG. 9.

FIG. 5 illustrates a system 500 managing the function of the image projecting units present in a medical imaging apparatus in accordance with an embodiment. The system 500 includes a projection control unit 502 configured to control the operation and functioning of the image projecting units such as the image projecting unit 310, the image projecting unit 312, the image projecting unit 314 and the image projecting unit 202. In an embodiment the projection control unit 502 may operate or instruct an image projecting unit to project one or more images and audio in response to receiving a command or instruction from the operator. For example when a subject enters the scanning room 302 the operator (e.g. the operator 400) sends instructions for operating image projecting units (i.e. the image projecting unit 310, the image projecting unit 312, and the image projecting unit 314). The instructions are received at the projection control unit 502 and sent to the image projecting units. The projection control unit 502 also selects the images for example a forest safari video to be projected by the image projecting units. The subject can view these images when they are in the scanning room and their attention gets deviated from the sounds generated by the medical imaging apparatus or the subject's anxiety is reduced. In an embodiment based on a position of the subject in the scanning room for instance subject lying on the table or any other location with the scanning room, the projection control unit 502 can activate or operate one or more image projecting units.

The images may be received from a video/audio device 504 and are transferred or transmitted to a selector/splitter 506. The video/audio device 504 may be wired or wirelessly connected to the selector/splitter 506. The selector/splitter 506 also receives instructions from the projection control unit 502 for selecting one or more image projecting units. The selector/splitter 506 transmits the received images to the selected image projecting units for projection. Considering the instance where the subject enters the scanning room the selector/splitter 506 selects the image projecting units (i.e. the image projecting unit 310, the image projecting unit 312, and the image projecting unit 314) and transmits the images to them for projecting to the walls and the ceiling. The selector/splitter 506 selects the image projecting units based on instructions from the projection control unit 502. Now when the subject lies on the table and passes through the image capturing subsystem of the medical imaging apparatus then the projection control unit 502 instructs the selector/splitter 506 to select an image projecting unit such as the image projecting unit 202. The selector/splitter 506 transmits the images to the image projecting unit 202 to project them onto an inner surface of the image capturing subsystem.

In an embodiment the images can be transferred from a plug/play device 508 that can be connected to the selector/splitter 506. The plug/play device 508 may include for example a mobile phone, a personal digital assistant (PDA), a laptop, a USB device, a tablet PC, an external hard drive, and a pad device. The selector/splitter 506 may receive images as input from an imaging device 510 i.e. a camera. The imaging device 510 may capture images of the technician performing the imaging procedure on the subject. The imaging device 510 may be positioned near to the technician so as to capture the images of the technician. The images may show the face of the technician. The technician can provide live instructions to the subject for performing the imaging procedure. This is further explained in conjunction with FIG. 9. The system 500 further includes a power source 512 supplying power to the image projecting units for their functioning. The power source 512 may be a direct power source or a battery power source.

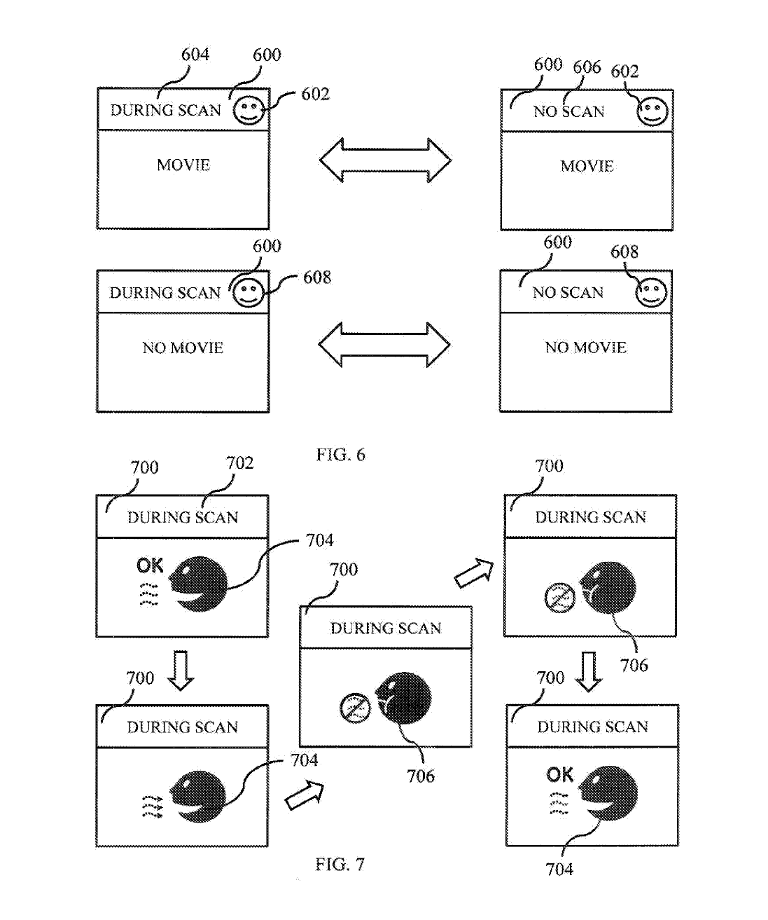

FIG. 6 is a schematic illustration of a user interface (UI) 600 presenting images for example movie during a scanning or imaging procedure and when scanning or imaging procedure is not performed in accordance with an exemplary embodiment. The UI 600 presents a movie along with an emoticon 602 while performing the scanning procedure. The UI 600 displays a message 604 showing “during scan” indicating scanning is being performed. The UI 600 may be projected at an inner surface of an image capturing subsystem of a medical imaging apparatus in case the subject is positioned within the image capturing subsystem. When scanning is not being performed the UI 600 presents a movie to the subject's view. The UI 600 also displays a message 606 showing “no scan” indicating scanning is not being performed. The movie may be projected onto the walls and the ceiling of the scanning room because the subject may be positioned outside the image capturing subsystem. In another scenario the UI 600 may not project any movie while performing the scanning procedure and when the scanning procedure is not being performed. The UI 600 also presents an emoticon 608 when the movie is not shown.

Turning now to FIG. 7 is a schematic illustration of a UI 700 visually providing instructions to the subject while performing an imaging or scanning procedure in accordance with an exemplary embodiment. The UI 700 may display a message 702 showing “during scan” indicating the scanning is being performed. The UI 700 may be displayed to the subject when positioned within the image capturing subsystem. The UI 700 may be projected on to the inner surface of the image capturing subsystem. At first stage an emoticon 704 may be presented in the UI 700 instructing the subject to breath. At the second stage the emoticon 704 is displayed as breathing to instruct the subject to continue breathing while scanning. At any point if the subject needs to hold breath, an emoticon 706 may be displayed indicating to stop breathing. The subject can hold breath at this stage. The emoticon 706 is presented to the subject through the UI 700 till when the technician may need the subject to hold the breath for forming the scanning procedure. When the technician does not require the subject to hold the breath then the emoticon 704 is presented again in the UI 700. The subject can start breathing freely in a relaxed manner. In an embodiment in addition to visually displaying the steps to be followed voice instructions may be also provided assisting the subject to follow the steps conveniently. It may be appreciated that emoticons and voice instructions may be used for providing instructions to the subject other than holding the breath of the subject without deviating away from the scope of this disclosure. Further it may be noted that emoticons and voice instructions may be used for conveying procedural instructions to the subject applicable for performing the imaging procedure.

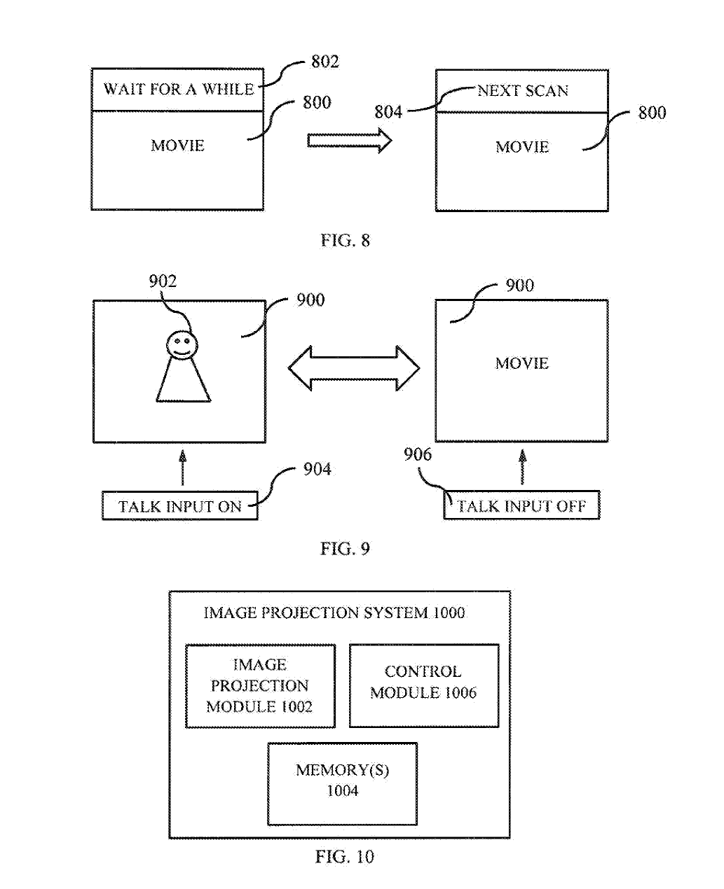

When a subject enters a scanning room for performing imaging, various pre-scan procedures may need to be performed. The pre-scan procedures are performed to prepare the medical imaging apparatus for performing the scanning or imaging of the subject. The subject needs to be intimated about this pre-scan procedure. FIG. 8 illustrates a UI 800 presenting a message 802 “wait for a while” along with a movie when the subject enters the scanning room in accordance with an exemplary embodiment. The UI 800 may be projected onto one or more walls and ceiling of the scanning room. This indicates that the subject needs to wait for a while for the pre-scan procedure to get completed. Thereafter the scanning procedure may start indicated by a message 804 showing “next scan” once the pre-scan procedure is completed. The subject can lie on a table of the medical imaging apparatus and scanning procedure can begin. As the movie is displayed throughout the subject may concentrate on the movie and anxiety of the subject may be reduced.

The technician may need to provide instructions to the subject while performing the imaging procedure. FIG. 9 illustrates a UI 900 displaying a face of a technician 902 in accordance with an exemplary embodiment. For instance the subject may be positioned within the image capturing subsystem of the medical imaging apparatus, then if the technician 902 may want to speak to the subject or give instructions to the subject, then the technician can enter a talk input command indicated by “talk input on” 904. This command may be received at the projection control unit 502 for activating an imaging device such as the imaging device 510 for capturing the face of the technician. The image of the technician i.e. a live video image of the face may be presented in the UI 900 projected onto the inner surface of the image capturing subsystem. The projecting control unit 502 activates one or more image projecting units for projecting the UI 900. The technician can give instructions to the subject to perform any steps that are needed as part of the imaging procedure. In another instance the instructions may be for example but not limited to, alerting the subject to stop unwanted movements or positioning correctly on the table for conducting imaging etc. As the face of the technician can be seen, a face to face interaction between the subject and the technician can be achieved. Similarly as explained earlier in conjunction with FIG. 3 an imaging device may be present within the image capturing subsystem for capturing the images of the subject. These images (i.e. live images) are presented to the technician for monitoring the position and movements of the subject.

Whenever the technician wants to stop displaying the face of the technician, the technician can input a command “talk input off” 906 which is received at the projection control unit 502. The projection control unit 502 may then stop receiving images from the imaging device such as the imaging device 510. Consequently a movie or other images may be presented to the subject. The subject can continue to watch these images or movie. In another instance no images may be projected to walls or ceiling or inner surface of the image capturing subsystem.

Now turning to FIG. 10 illustrating a block diagram of an image projection system 1000 in accordance with an embodiment. The image projecting system 1000 includes an image projection module 1002 configured to control projection of images one or more walls of a scanning room and an inner surface of the image capturing subsystem. These projected images are viewed by the subject and deviates the attention of the subject from the sounds generated from the medical imaging apparatus to the images. The image capturing subsystem can facilitate the subject to pass therethrough for capturing the medical images of the subject. The image projection system 1000 also includes one or more memory(s) 1004 communicably coupled to the image projection module 1002. In an embodiment the one or more memory(s) 1004 may store the images and then transmit them to the image projecting units. The image projection module 1002 is also configured to present audio along with the images. The audio may be presented synchronous with the images. This will enable the images to be understood by the subject. The images may include but not limited to, video images, static images, animated images and procedural images. A control module 1006 present in the image projection system 1000 may be configured to control communication of the images to the image projection module 1002.

The control module 1006 may operate or instruct an image projecting unit to project one or more images and audio in response to receiving a command or instruction from the operator. For example when a subject enters a scanning room, an operator (e.g. the operator 400) sends instructions for operating image projecting units. The instructions are received at the control module 1006 and sent to the image projecting units. The control module 1006 also selects the images for example a forest safari video to be projected by the image projecting units. The subject can view these images when they are in the scanning room and their attention gets deviated from the sounds generated by the medical imaging apparatus or the subject's anxiety is reduced. In an embodiment based on a position of the subject in the scanning room for instance subject lying on the table or any other location with the scanning room, the control module 1006 can activate or operate one or more image projecting units.

The control module 1006 is also configured to communicate with an imaging device for example a camera in the image capturing subsystem of the medical imaging apparatus. The imaging device captures images of the subject. These images indicate the activities and movements of the subject within the image capturing subsystem. The control module 1006 manages the transmission of these images to the operator or the technician of the medical imaging apparatus. The technician can view these images and accordingly instructions can be given by the technician to the subject. For example a technician can alert or give instructions to the subject to stop unwanted movements of the subject in the image capturing subsystem. The unwanted movements can be identified using the images received from the imaging device present in the image capturing subsystem.

The control module 1006 displays messages on to one or more of the walls and the inner surface of the image capturing subsystem for the subject to view. The message may be displayed on an UI projected. The UI displays a message showing “during scan” indicating scanning is being performed. The UI may be projected at an inner surface of an image capturing subsystem of a medical imaging apparatus in case the subject is positioned within the image capturing subsystem. When scanning is not being performed the UI presents a movie to the subject's view. The UI also displays a message showing “no scan” indicating scanning is not being performed. The movie may be projected onto the walls and the ceiling of the scanning room because the subject may be positioned outside the image capturing subsystem. In another scenario the UI may not project any movie while performing the scanning procedure and when the scanning procedure is not being performed. The UI also presents an emoticon when the movie is not shown. It may be appreciated that the control module 1106 may be configured to project different messages that need to be communicated to the subject.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The methods described in conjunction with figures can be performed using a processor or any other processing device. The method steps can be implemented using coded instructions (e.g., computer readable instructions) stored on a tangible computer readable medium. The tangible computer readable medium may be for example a flash memory, a read-only memory (ROM), a random access memory (RAM), any other computer readable storage medium and any storage media. Although the method of projecting images onto one or more walls and a ceiling using in-built image projecting units in a medical imaging apparatus is explained with reference to the flow chart of figures, other methods of implementing the method can be employed. For example, the order of execution of each method steps may be changed, and/or some of the method steps described may be changed, eliminated, divide or combined. Further the method steps may be sequentially or simultaneously executed for projecting images onto one or more walls and a ceiling using in-built image projecting units in a medical imaging apparatus.

This written description uses examples to disclose the invention, including the best mode, and also to enable any person skilled in the art to practice the invention, including making and using any computing system or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A medical imaging apparatus, comprising:
an image capturing subsystem configured to capture medical images of a subject; and
an image projecting unit connected to the image capturing subsystem and configured to project an image for the subject to view, wherein the image is projected to at least one of a scanning room wall or an inner surface of the image capturing subsystem.

2. The medical imaging apparatus of claim 1, wherein the image is projected while capturing the medical images of the subject.

3. The medical imaging apparatus of claim 1, wherein the image comprises at least one of video images, static images, animated images, procedural images, or a combination thereof, wherein the procedural images are associated with a set of instructions to be followed by the subject.

4. The medical imaging apparatus of claim 3, wherein the image comprises two dimensional images and three dimensional images.

5. The medical imaging apparatus of claim 3, further comprising a projection control unit configured to present audio along with the image, wherein the audio is presented synchronous with the image.

6. The medical imaging apparatus of claim 5, wherein the projection control unit is further configured to:
receive a command from an operator of the medical imaging apparatus; and
present at least one of the at least one image and the audio to the subject in response to receiving the command.

7. The medical imaging apparatus of claim 5, wherein the projection control unit is further configured to project the image for the subject to view in response to capturing the medical images of the subject.

8. The medical imaging apparatus of claim 5, wherein the at least one image capturing unit is configured at a predefined location in the image capturing subsystem.

9. The medical imaging apparatus of claim 7, wherein the projection control unit is configured to manage the transmission of the images of the subject for display to an operator of the medical imaging apparatus.

10. The medical imaging apparatus of claim 5, wherein the projection control unit is further configured to display messages on at least one of the walls and the inner surface of the image capturing subsystem for the subject to view.

11. The medical imaging apparatus of claim 5 further comprises a shielding component for covering the projection control unit.

12. An image projection system communicably coupled to a medical imaging apparatus, the image projection system, comprising:
an image projection module for controlling projection of images for a subject to view, wherein at least one image is projected to at least one of walls of a scanning room and an inner surface of an image capturing subsystem of the medical imaging apparatus, the image capturing subsystem is configured to facilitate the subject to pass therethrough for capturing medical images of the subject; and
at least one memory communicably coupled to the image projection module.

13. The image projection system of claim 12, wherein the image projection module is further configured to present audio along with the images, wherein the audio is presented synchronous with the images.

14. The image projection system of claim 12 further comprises a control module communicably coupled to the image projection module, the control module configured to:
process a command from an operator of the medical imaging apparatus; and
control presentation of at least one of the at least one image and the audio to the subject in response to receiving the command.

15. The image projection system of claim 14, wherein the control module is further configured to:
control communication of the images to the image projection module; and
manage transmission of the images of the subject for display to an operator of the medical imaging apparatus.

16. The image projection system of claim 14, wherein the image projection module is configured to display messages on at least one of the walls and the inner surface of the image capturing subsystem for the subject to view.

17. The image projection system of claim 15, wherein the control module is further configured to instruct the image projection module to project the images for the subject to view in response to capturing the medical images of the subject.

18. A method for communicating with a subject during a medical imaging exam, comprising:
providing an image capturing subsystem positioned within a scanning room and the image capturing subsystem comprising an image projecting unit; and
projecting with the image projecting unit an image on at least one of a scanning room wall.

19. The method of claim 18, wherein the image is one of a video image, a static image, an animated image, or a combination thereof.

20. The method of claim 18, wherein the image is a procedural image associated with a set of instructions to be followed by the subject.

21. The method of claim 18, further comprising presenting audio with the projecting step.
